(19) United States
(12) Patent Application Publication
(10) Pub. No.: US 2021/0338359 A1
(43) Pub. Date: Nov. 4, 2021

(54) SHARPS MEDICAL ADHERENCE LID

(71) Applicant: HealthBeacon Ltd., Dublin 12 (IE)

(72) Inventors: James JOYCE, Needham, MA (US); Richard SHATTOCK, Dublin (IE)

(21) Appl. No.: 17/273,537

(22) PCT Filed: Sep. 5, 2018
(86) PCT No.: PCT/IB2018/000993

§ 371 Date: Mar. 4, 2021

(51) Int. Cl. A61B 50/36; G16H 20/17; G16H 40/20; G16H 40/67; A61B 90/90; A61B 90/00

(52) U.S. Cl. CPC A61B 50/362; G16H 20/17; G16H 40/20; A61B 2050/0074; A61B 90/90; A61B 90/08; G16H 40/67

(57) ABSTRACT

Methods and apparatus for a sharps medical adherence lid. A sharps medical adherence lid includes a housing, and a clasping device, the clasping device configured to removably attach the housing to a medical sharps bin, the housing including an upper latch, a spring loaded hinged door secured by the upper latch, a slide adapted to capture an object passing through the spring loaded hinged door, a lower opening configured to receive the object from the slide and enable it to pass in the medical sharps bin.

### BACKGROUND OF THE INVENTION

The present invention relates generally to sharps bins, and more particularly to a sharps medical adherence lid.

Traditional medical waste in the form of a needle and/or a syringe requires safe and regulated disposal in most jurisdictions. Many injectable medications when provided to a patient for administration in a home setting include the provision of a disposable medical waste container or sharps bin to enable the disposal and safe storage of the sharps component following administration. In general, a sharp is any device having corners, edges, or projections capable of cutting or piercing the skin, such as a needle of syringe.

Typical sharps containers are passive and have no electronic components to enable the detection of an object being dropped into the container. The ability to capture this event represents a close proxy to when administration of the medication actually occurred in the home.

Once a patient is prescribed an injectable drug, they are typically trained in the administration of the therapy in the home setting. A nurse will visit and discuss the patient's illness and support infrastructures that often accompany the prescription drug as well as train them how to safely administer and dispose of their used injectable into the sharps container. Once the nurse leaves, the onus is on the patient to follow their unique injection schedule and administer their medication according to their Physicians instructions. Studies demonstrate that adherence to self-administered medication can drop dramatically in the early stages of a patient starting their medication and results estimate that only between 30-50% of patients will actually be adherent to their medication. Physicians do not have any visibility on their patient's injection habits and rely on their word to determine that they are taking their medication correctly.

The ability to convert a passive sharps container into a smart connected device allows several intervention opportunities to ensure non-adherent patients can be returned to their prescription schedule quickly or provide opportunities for medical intervention to ensure the best clinical outcome for the patient. For example, where a patient does not drop an injectable device into a sharps bin when they are scheduled to will result in a Smart SMS reminder to relevant parties, which could include the Patient, the Carer, the Healthcare Professional's or third party vendors to ensure the appropriate course of action can be taken to improve clinical outcome.

### SUMMARY OF THE INVENTION

The following presents a simplified summary of the innovation in order to provide a basic understanding of some aspects of the invention. This summary is not an extensive overview of the invention. It is intended to neither identify key or critical elements of the invention nor delineate the scope of the invention. Its sole purpose is to present some concepts of the invention in a simplified form as a prelude to the more detailed description that is presented later.

The present invention provides methods and apparatus for a sharps adapter medical adherence lid.

In general, in one aspect, the invention features a sharps medical adherence lid including a housing, one or more clasping members, the one or more clasping members configured to removably attach the housing to a medical sharps bin, the housing including an upper latch, a spring loaded hinged door secured by the upper latch, a slide adapted to capture an object passing through the spring loaded hinged door, and a lower opening configured to receive the object from the slide and enable it to pass in the medical sharps bin.

In another aspect, the invention features a method including providing a sharps bin, providing an adherence lid, removably attaching the adherence lid to the sharps bin with a locking mechanism, configuring the adherence lid to communicate with a remote server, receiving an object through a gate in the adherence lid, recording an event representing an object passing through the gate in the adherence lid, passing the object from the gate to the sharps bin, and sending the recorded event to the remote server.

Embodiments of the invention may have one or more of the following advantages.

The present invention enables any medical waste container or sharps container to become a connected sharps bin.

Data captured by the lid on a medical waste container is communicated to a customer relationship management (CRM) system and reconciled to a patient's health record and prescription schedule.

These and other features and advantages will be apparent from a reading of the following detailed description and a review of the associated drawings. It is to be understood that both the foregoing general description and the following detailed description are explanatory only and are not restrictive of aspects as claimed.

### BRIEF DESCRIPTION OF THE DRAWINGS

The invention will be more fully understood by reference to the detailed description, in conjunction with the following figures, wherein:

FIG. 1 is a block diagram of an exemplary medical adherence system.

FIG. 2 is flow diagram.

### DETAILED DESCRIPTION

The subject innovation is now described with reference to the drawings, wherein like reference numerals are used to refer to like elements throughout. In the following description, for purposes of explanation, numerous specific details are set forth in order to provide a thorough understanding of the present invention. It may be evident, however, that the present invention may be practiced without these specific details. In other instances, well-known structures and devices are shown in block diagram form in order to facilitate describing the present invention.

As used in this application, the term “or” is intended to mean an inclusive “or” rather than an exclusive “or.” That is, unless specified otherwise, or clear from context, “X employs A or B” is intended to mean any of the natural inclusive permutations. That is, if X employs A, X employs B, or X employs both A and B, then “X employs A or B” is satisfied under any of the foregoing instances. Moreover, articles “a” and “an” as used in the subject specification and annexed drawings should generally be construed to mean “one or more” unless specified otherwise or clear from context to be directed to a singular form.

Adherence is a concern with biological drugs with high numbers of patients (30-50%) demonstrating non-compliance with their prescribed regime. The adherence lid of the present invention provides the ability to monitor the interaction with a sharps bin and represents a close proxy to the administration event.

Failure to interact with the bin can signal an action event, sending a text reminder to the patient, to a family member or nurse. The notification event can lead to an intervention event and hopefully the patient taking their medication. The patient may not take their medication, possibly due to illness or traveling, but the ability to see if the interaction occurred or not allows the automated notification procedure and a follow up by the relevant HealthCare professional to ensure that the patient gets back on their medication schedule as quickly as possible.

Reports of a patient's adherence can be sent on a regular basis to the Nurse or Doctor which can be accessed via Healthcare professional app or via a unique portal login or other reporting platform. This allows them to check a patient's adherence to their medication schedule prior to any visits and allows an informed discussion around the patient's compliance to their medication schedule. A patient not compliant with little improvement in their clinical symptoms would likely be advised to stay on their medication and try and improve their injection habits. A patient with good adherence and good clinical outcome would also be advised to stay on the medication as the drug is working, but a patient with good adherence and zero improvement may be advised to switch their medication (this would depend on how long they have been taking the medication as often patients need to be persistent for several months to note any improvements). Either way patients and doctors can make informed decisions.

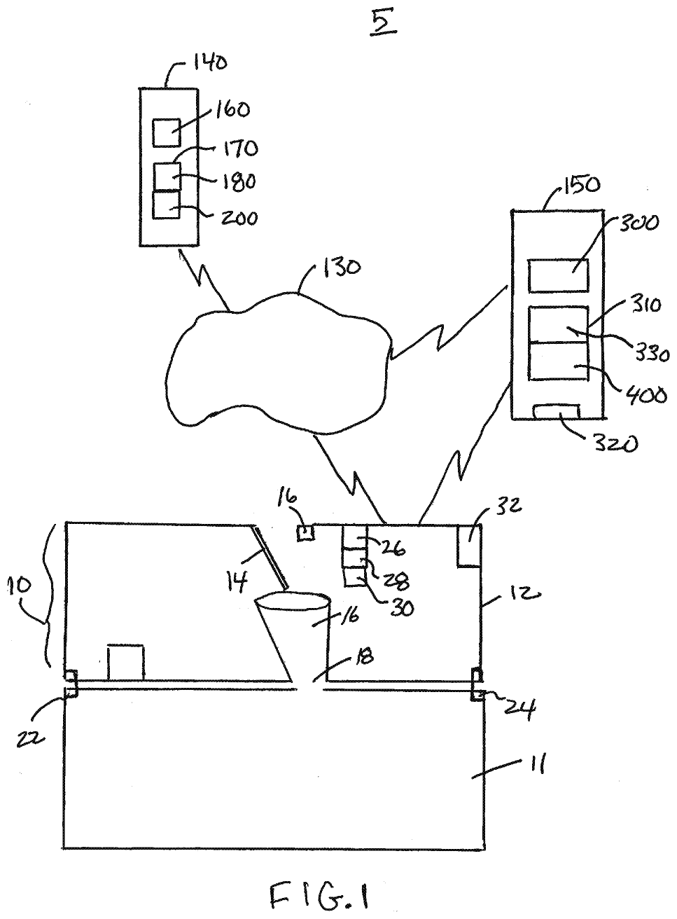

As shown in FIG. 1, an exemplary medical adherence system 5 includes a sharps medical adherence lid 10 secured to a sharps bin 11. The sharps medical adherence lid 10 includes a housing 12. The housing 12 includes a spring loaded hinged door 14 secured by a latch 16. The spring loaded hinged door 14 opens to an upper portion of a slide 16. A bottom portion of the slide 16 includes an lower opening 18. The housing 12 also includes one or more clasping members 22, 24, that enable the housing 12 to be removably secured a top portion of the sharps bin 11. In one embodiment, the sharps bin 11 is housed within a decorative enclosure and the lid 10 secured affixed to both the sharps bin 11 and the decorative enclosure.

The housing 12 also may include one or more sensors 26, a camera 28, a sim card and/or other Near Field Communication Device (e.g., WiFi, Bluetooth, RFID, and so forth) 30, an antenna 32 for wireless communication and a power supply 34. The power supply 34 may be battery powered or AC powered. When the latch 16 is released and the hinged door opens, passing into the slide 16 of any object, such as medical waste (e.g., syringe), results in the event being logged by the one or more sensors 26, the object photographed by the camera 28 and event time stamped, thus providing a record of the event. This event record, sometimes referred herein as adherence data, may then be transmitted by the antenna 32 to a server 140 that may be part of a network 130 of interconnected computers (e.g., the Internet). A smartphone 150 may also be communicatively linked to the medical adherence system 5.

The server 140 includes at least a processor 160 and a memory 170. The memory 170 includes at least an operating system (O/S) 180 and a medical adherence process 200. The medical adherence process 200 is configured to receive and process adherence data from the sharps medical adherence lid 10.

The smartphone 150 includes at least a processor 300 and a memory 310. The memory 310 includes at least an operating system (O/S) 330 and a mobile medical adherence application (app) 400. The mobile medical adherence app 400 is configured to receive alerts from the medical adherence process 200. An example alert is a message indicating that a user (e.g., patient) is scheduled for an injection using a syringe. In one embodiment, as the message is received by the user, medical adherence process 200 sends a signal to the sharps medical adherence lid 10 that turns on a reminder light (not shown) on the housing 12. This reminder light is turned off once the spring loaded hinged door 14 opens.

In one embodiment, the medical adherence process 200 is a customer relationship management (CRM) system that reconciles the received adherence data to the patient's health record and prescription schedule. Using machine learning, the medical adherence process 200 enables a calculation of a patient's personal medical adherence score depending on compliance with their prescription schedule. Moreover, the medical adherence process 200 is configured to recognize patient behavior, disposal and injection habits and, in combination with additional captured data via Near Field Communications (NFC), WiFi or other connected devices, including data input by the patient through their mobile medical adherence app 400, the medical adherence process 200 can provide a series of notifications or actionable events for the patient and/or third parties.

In one embodiment, when a patient is nearing completion of their stock medication, a notification will be sent to their prescribing pharmacy, triggering a dispensing event via the pharmacy's CRM system. In another embodiment, where a patient requires a new prescription or physician appointment the medical adherence process 200 can communicate notifications as appropriate.

In other embodiments, the medical adherence process 200 is configured to analyze, summarize, and/or profile the received data for patient behaviors, possible medical interventions, and so forth. In addition, reports generated by the medical adherence process 200 may be used to support clinician and patient dialogue around adherence. In embodiments, the medical adherence process 200 presents patient and clinician dashboards specifically targeted at a patient, a doctor and/or a pharmacist.

In other embodiments, the medical adherence process 200 is configured to analyze and determine the capacity of the sharps bin. Notifications can be configured and sent to the necessary third party vendor to resupply a new sharps bin and allow the safe exchange and disposal of the full sharps bin.

In another embodiment, the medical adherence process 200 is configured to determine when a new bin has been inserted or the lid placed on top of a empty sharps container. The patient's injection schedule is automatically updated to allow the maximum number of used injectables to be deposited into the bin before capacity is met and a new notification is sent a for replacement bin.

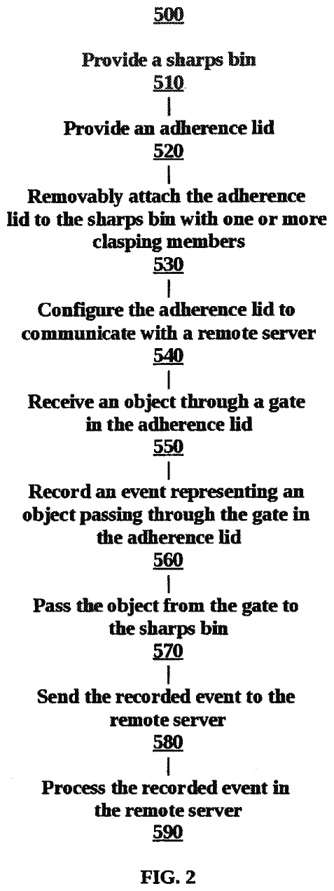

As shown in FIG. 2, a process 500 includes providing (510) a sharps bin, providing (520) an adherence lid and removably attaching (530) the adherence lid to the sharps bin with a locking mechanism.

Process 500 configures (540) the adherence lid to communicate with a remote server.

Process 500 receives (550) an object through a gate in the adherence lid.

Process 500 records (560) an event representing an object passing through the gate in the adherence lid.

Process 500 passes (570) the object from the gate to the sharps bin.

Process 500 sends (580) the recorded event to the remote server.

Process 500 processes (590) the recorded event in the remote server.

In another embodiment, a sharps medical adherence lid includes a housing, a communication device, an internal recess or chamber, an access door, the chamber adapted to capture an object being placed in it, and a lower opening adapted to pass an object through it into a medical sharps bin.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed is:

1. A sharps medical adherence lid comprising:
a housing;
a locking mechanism configured to removably attach the housing to a medical sharps bin, the housing comprising:
an upper latch;
a spring loaded hinged door secured by the upper latch;
a slide adapted to capture an object passing through the spring loaded hinged door; and
a lower opening configured to receive the object from the slide and enable it to pass in the medical sharps bin.

2. The sharps medical adherence lid of claim 1 wherein the housing further comprises:
one or more sensors, the one or more sensors configured to detect the object and assign a date and time stamp.

3. The sharps medical adherence lid of claim 2 wherein the housing further comprises:
a camera, the camera configured to photograph the object.

4. The sharps medical adherence lid of claim 3 wherein the camera is detachable.

5. The sharps medical adherence lid of claim 3 wherein the housing further comprises:
a power supply or battery.

6. The sharps medical adherence lid of claim 5 wherein the housing further comprises:
a sim card; and
an antenna, the antenna used to transmit and receive signals from a remote server and a smartphone.

7. The sharps medical adherence lid of claim 6 wherein the housing further comprises:
a light, the light controlled by signal received from the remote server.

8. The sharps medical adherence lid of claim 1 wherein the housing further comprises one or more mirrors to determine the capacity of the internal sharps bin.

9. The sharps medical adherence lid of claim 1 wherein the housing further comprises one or more sensors to determine when the internal sharps container has been removed or exchanged.

10. A method comprising:
providing a sharps bin;
providing an adherence lid;
removably attaching the adherence lid to the sharps bin with a locking mechanism;
configuring the adherence lid to communicate with a remote server;
receiving an object through a recess in the adherence lid;
recording an event representing the object passing through the recess in the adherence lid;
passing the object from the recess to the sharps bin; and
sending the recorded event to the remote server.

11. The method of claim 10 wherein the object is selected from the group consisting of a passive syringe, a connected syringe, an injectable device and a smart packaged device.

12. The method of claim 10 further comprising processing the recorded event in the remote server.

13. The method of claim 10 wherein the adherence lid comprises:
one or more sensors;
a camera;
a sim card;
an antenna for wireless communication; and
a power supply.

14. The method of claim 10 wherein recording comprises:
assigning a date; and
assigning a time stamp.

15. The method of claim 14 wherein recording further comprises:
capturing a photograph of the object.

16. The method of claim 12 wherein processing the recorded event comprises:
reconciling a time and date associated with the object to a patient's health record and prescription schedule.

17. The method of claim 16 wherein processing the recorded event further comprises:
calculating the patient's personal adherence score, the personal adherence score representing a percentage compliance with respect to the patient's prescription schedule.

18. The method of claim 16 wherein processing the recorded event further comprises:
sending a prescription refill notification to a pharmacy system if the object represents one of a few remaining objects dictated by the prescription schedule.

19. The method of claim 16 wherein processing the recorded event further comprises:
sending a notification to a communication device or server to re-align the patient with their medication schedule.

20. The method of claim 16 wherein processing the recorded event further comprises:
receiving a patient reported outcome via a communication device and reconciling the received patient reported outcome to a digital record.

21. A sharps medical adherence lid comprising:
a housing;
a communication device;
an internal recess or chamber, the chamber adapted to capture an object being placed in it;
an access door; and
a lower opening adapted to pass the object through it into the medical sharps bin.
